(19) United States
(12) Patent Application Publication
(10) Pub. No.: US 2005/0228397 A1
(43) Pub. Date: Oct. 13, 2005

(54) Cavity filling device

(76) Inventors: Hugues F. Malandain, Mountain View, CA (US); Richard W. Layne, Sunnyvale, CA (US); Alberto Ruiz Cantu, San Francisco, CA (US); Aaron D. Markworth, Saddle Brook, NJ (US); Robert M. Scribner, Niwot, CO (US); Michael L. Reo, Redwood City, CA (US); Mark A. Reiley, Piedmont, CA (US); Ryan P. Boucher, San Francisco, CA (US)

Correspondence Address: FISH & RICHARDSON P.C., PO BOX 1022, MINNEAPOLIS, MN, 55440-1022, US

(21) Appl. No.: 10/979,781

(22) Filed: Nov. 1, 2004

Related U.S. Application Data
(63) Continuation-in-part of application No. 10/617,976, filed on Jul. 11, 2003.
Application No. 10/617,976 is a division of application No. 09/804,107, filed on Mar. 12, 2001, now Pat. No. 6,613,054.
Application No. 09/804,107 is a division of application No. 09/134,323, filed on Aug. 14, 1998, now Pat. No. 6,241,734.

(51) Int. Cl. A61B 17/58

(52) U.S. Cl. 606/93

(57) ABSTRACT

A method and apparatus for filling a cavity in a patient's body with a material are provided. The apparatus can include a flexible tube, a barrel and a plunger. The flexible tube has a first end, a second end, and a lumen extending from the first end to the second end and operable to hold a material for conveyance through the first end into a cavity. The barrel is in fluid communication with the second end of the flexible tube and includes a lumen configured to receive material for delivery into the flexible tube. The plunger is configured to advance material through the lumen in the barrel and into the flexible tube. In one implememtation, there is substantially a one to one ratio of advancement of the plunger to a volume of material advanced from the rigid first end of the flexible tube.

### CROSS REFERENCE TO RELATED APPLICATIONS

This application is a continuation-in-part of, and claims priority to, pending U.S. application Ser. No. 10/617,976, filed on Jul. 11, 2003, which is a divisional application of U.S. application Ser. No. 09/804,107, filed Mar. 12, 2001, now U.S. Pat. No. 6,613,054 issued Sep. 2, 2003, to Scribner, et al, which is a divisional application of U.S. application Ser. No. 09/134,323, filed Aug. 14, 1998, now U.S. Pat. No. 6,241,734 issued Jun. 5, 2001, Scribner et al. Priority is claimed to the above referenced applications and the contents of the above referenced applications are hereby incorporated by reference herein in their entirety.

### TECHNICAL FIELD

This invention relates to medical methods and apparatus.

### BACKGROUND

A vertebral compression fracture (VCF) can occur when a vertebral body is too weak to support a load and the spine collapses. A VCF may cause the spine to shorten, leading to spinal deformities and altering spinal biomechanics. Collapse may result in thoracic and lumbar spinal deformity and is often seen in elderly people. The spinal deformity, commonly known as a Dowager's Hump, is also referred to as kyphosis. Several causes can lead to a VCF, including osteoporosis, cancer or a traumatic incident, such as a fall or car accident.

A treatment for a VCF can involve injecting a material into vertebra, either at low or high pressure. Optionally, a surgical balloon can first be inserted into a vertebra and expanded to restore a collapsed vertebra to its original shape. A material can then be inserted into the restored vertebra, which, upon hardening, can maintain the original shape of the vertebra. The technique of inserting a material into a cavity, such as a collapsed vertebra, can also be used to treat other medical conditions, for example, in knee or hand joints.

### SUMMARY

This invention relates to a method and apparatus for filling a cavity in a patient's body with a material. In general, in one aspect, the invention features an apparatus including a flexible tube, a barrel and a plunger. The flexible tube has a rigid first end, a second end, and a lumen extending from the first end to the second end and operable to hold a material for conveyance through the rigid first end into a cavity. The barrel is in fluid communication with the second end of the flexible tube and includes a lumen configured to receive material for delivery into the flexible tube. The plunger is configured to advance material through the lumen in the barrel and into the flexible tube. There is substantially a one to one ratio of advancement of the plunger to a volume of material advanced from the rigid first end of the flexible tube.

In general, in another aspect, the invention features an apparatus including a flexible tube, a barrel and a plunger. The flexible tube has a rigid first end, a second end, and a lumen extending from the first end to the second end and operable to hold a material for conveyance through the rigid first end and into a cavity. The barrel is in fluid communication with the second end of the flexible tube and includes a lumen configured to receive material for delivery into the flexible tube. The barrel's lumen has a diameter substantially the same as a diameter of the flexible tube's lumen. The plunger is configured to advance material through the lumen in the barrel and into the flexible tube.

In general, in another aspect, the invention features an apparatus including a flexible tube, a barrel and a plunger. The flexible tube has a first end and a second end, and a lumen extending from the first end to the second end and operable to hold a material for conveyance through the first end. The barrel is in fluid communication with the second end of the flexible tube and includes a lumen configured to receive material for delivery into the flexible tube. The plunger is configured to advance material through the lumen in the barrel and into the flexible tube.

In general, in another aspect, the invention features an apparatus including a flexible tube, a barrel, a cartridge and a plunger. The flexible tube has a rigid first end, a second end, and a lumen extending from the first end to the second end and operable to hold a material for conveyance through the rigid first end into a cavity. The barrel is in fluid communication with the second end of the flexible tube and includes a lumen configured to receive a cartridge pre-loaded with material for delivery into the flexible tube. The cartridge is configured for insertion into the barrel and includes a lumen configured to receive material for delivery into the flexible tube. The plunger is configured to advance material through the lumen in the cartridge and into the flexible tube, such that there is substantially a one to one ratio of advancement of the plunger to a volume of material advanced from the rigid first end of the flexible tube.

Implementations can include one or more of the following features. The apparatus can further include a cannula having a first end configured to receive the first end of the flexible tube and having a second end configured to provide the first end of the flexible tube access to the cavity. The lumen of the flexible tube can be pre-loaded with the material, and the plunger can be operable to advance material through the lumen in the barrel into the flexible tube. The pre-loaded material in the flexible tube is thereby advanced through the lumen and into the cavity.

The apparatus can further include a connector (e.g., a luer fitting) connecting the flexible tube to the barrel, where the flexible tube is connected to the barrel such that a longitudinal axis of the barrel is at an angle in the range of 0° to 90° to a longitudinal axis of the flexible tube. In one implementation, the angle is substantially 40° to 50°. The barrel can include threads on an end of the lumen and the connector can further include threads configured to mate with the threads on the barrel. An assembly of the flexible tube connected to the barrel can be pre-loaded with the material, where the plunger is operable to advance at least some of the pre-loaded material into the cavity.

The barrel can further include a plurality of lumens disposed in the barrel and configured to receive material for delivery into the flexible tube. The barrel can be rotatable about a longitudinal axis of the barrel, and each lumen of the plurality of lumens can be preloaded with material. Once a lumen has been emptied into the flexible tube by action of the plunger, the plunger can be withdrawn, the barrel can be rotated and the plunger can engage a next lumen to fill the flexible tube.

The apparatus can further include a cartridge configured to receive material for delivery into the flexible tube, where the barrel's lumen is configured to receive the cartridge and to receive the material via the cartridge, and the plunger is configured to advance material through the lumen in the barrel via advancement through the cartridge.

The apparatus can include a plurality of cartridges configured for insertion into the barrel, each cartridge including a lumen configured to receive material for delivery into the flexible tube. The barrel can further include a plurality of lumens configured to receive a plurality of cartridges. The barrel is rotatable about a longitudinal axis of the barrel and each lumen can be loaded with a cartridge pre-loaded with material, such that once a cartridge has been emptied into the flexible tube, the barrel can be rotated and a next cartridge used to fill the flexible tube.

Implementations of the invention can realize one or more of the following advantages. The cavity filling assembly can allow an operator of the assembly to perform a cavity filling operation, while keeping the operator's hands outside of an x-ray field created by an imaging device required to assist the operator in maneuvering the assembly during the operation. For example, in a kyphoplasty procedure to fill a vertebra with bone filling cement, a C-arm imaging device is typically used to provide the operator with instantaneous imaging information necessary for the operator to perform a cavity filling operation. Preferably, the operator is able to manipulate the necessary cavity filling apparatus throughout the kyphoplasty procedure while only minimally exposing, if at all, his or her own body to an x-ray field created by the C-arm imaging device. The cavity filling assembly described herein is configured to allow the operator to keep his or her hands a distance from the C-arm imaging device while performing a cavity filling operation.

The cavity filling assembly can be configured so as to be positioned during a cavity filling operation such that a material can be advanced through the assembly without interfering with an imaging device also required to perform the operation. For example, in a kyphoplasty procedure, a C-arm imaging device is typically used as discussed above. There can be relatively little clearance between the C-arm imaging device and the patient's body. The cavity filling assembly includes a nozzle assembly that can be inserted into a cannula that has been inserted into the patient's body. A barrel attached to the nozzle assembly can be used to receive the cavity-filling material and operate as a handle for the operator. A connector between the nozzle assembly and the barrel can be configured to connect the nozzle assembly to the barrel at a range of different angles. For example, the angle can be selected to accommodate the clearance between an imaging device and a patient's body, while maintaining a suitable flow path for the material through the cavity filling assembly.

The cavity filling assembly can be configured to allow all of the cavity filling material necessary for a cavity filling operation to be loaded into the assembly at one time, so that iterative loading procedures are not required during the cavity filling operation. For example, a barrel including multiple chambers is described, where each chamber can be loaded with a cavity filling material, either directly or by inserting a loaded cartridge into the chamber. By providing multiple chambers, the barrel can be loaded at the start of the operation with sufficient material to completely fill the cavity, thereby facilitating the procedure.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A shows a perspective view of a cavity filling assembly.

FIG. 1B shows a perspective view of a nozzle assembly of the cavity filling assembly of FIG. 1A.

FIG. 1C shows a perspective view of a barrel of the cavity filling assembly of FIG 1A.

FIG. 2 shows the cavity filling assembly of FIG. 1A inserted into a cannula that has been positioned in a patient's body cavity.

FIG. 3A shows a plunging assembly within a cartridge.

FIG. 3B shows the plunging assembly and cartridge of FIG. 3A inserted within the cavity filling assembly of FIG. 1A.

FIG. 4A shows a side view of a connector for a cavity filling assembly.

FIG. 4B shows a perspective view of the connector of FIG. 4A.

FIG. 5 shows a front view of a cement mixer.

FIG. 6 is a flowchart showing a process for filling a cavity with a material using a cavity filling assembly.

FIG. 7 shows a multi-chambered barrel assembly.

FIGS. 8A-C show an alternative implementation of a multi-chambered barrel assembly.

Like reference symbols in the various drawings indicate like elements.

### DETAILED DESCRIPTION

An apparatus and method is described for injecting a material into a cavity in a patient's body. For illustrative purposes, the apparatus and method shall be described in the context of injecting a bone filling cement into a vertebra of a patient to treat kyphosis, although the apparatus and methods can be used to treat other conditions.

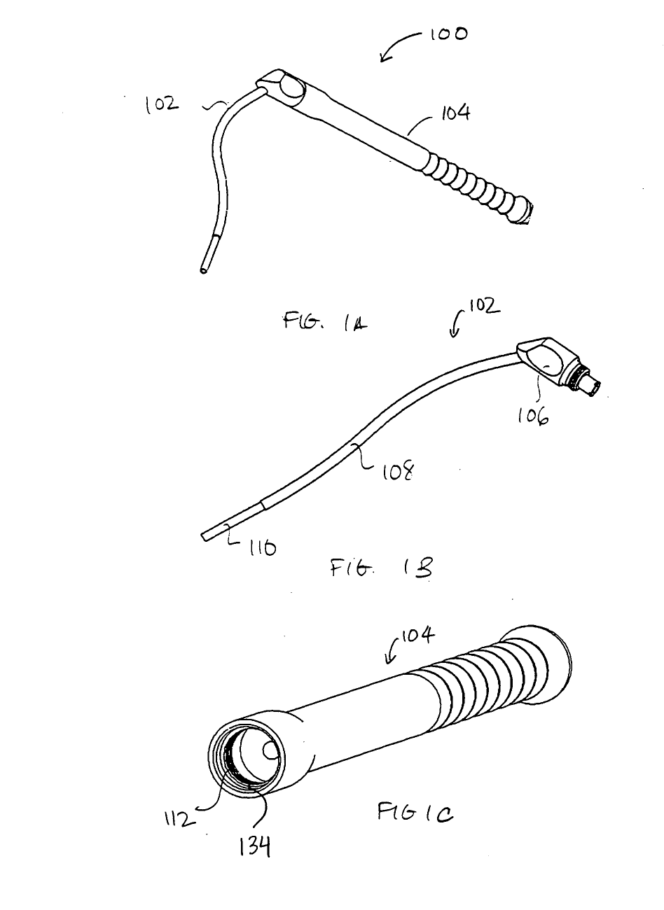

FIGS. 1A-1C show an apparatus that can be used to inject a material into a cavity, such as a vertebra. In the implementation shown, a cavity-filling assembly 100 includes a nozzle assembly 102 and a barrel 104. Referring to FIG. 1B, the nozzle assembly 102 is shown and includes a connector 106 attached to a flexible tube 108 terminating in a rigid distal end 110. Referring to FIG. 1C, the barrel 104 includes a hollow interior and a fitting 112 configured to mate with the connector 106 of the nozzle assembly 102.

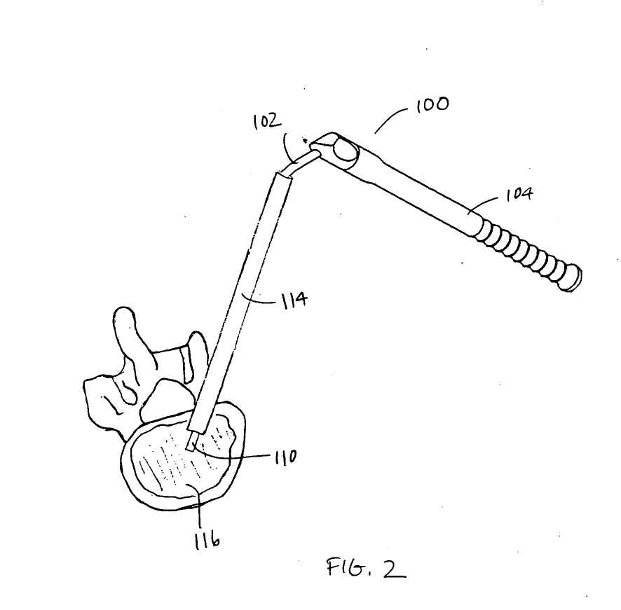

Referring to FIG. 2, the cavity-filling assembly 100 is shown with the nozzle assembly 102 inserted into a cannula 114. The cannula 114 has been inserted into the cavity 116 within the patient's body that is to receive a cavity filling material. For example, the cavity 116 shown is a vertebra, which may or may not have been previously restored to an original shape using a surgical balloon. A small incision in the patient's back can be used to insert the cannula 114 into the cavity 116. The nozzle assembly 102 can then be inserted into the cannula 114 to provide the distal end 110 access to the cavity 116. Examples of cavity filling material include: bone cement (e.g., polymethyl methacrylate (PMMA) cement, ceramics); human bone graft (e.g., autograft, allograft); and synthetic derived bone substitutes (e.g., calcium sulfate, calcium phosphate, hydroxyapatite). Cavity filling materials can be supplemented with other therapeutic substances, e.g., antibiotics, growth factors and chemotherapeutic agents.

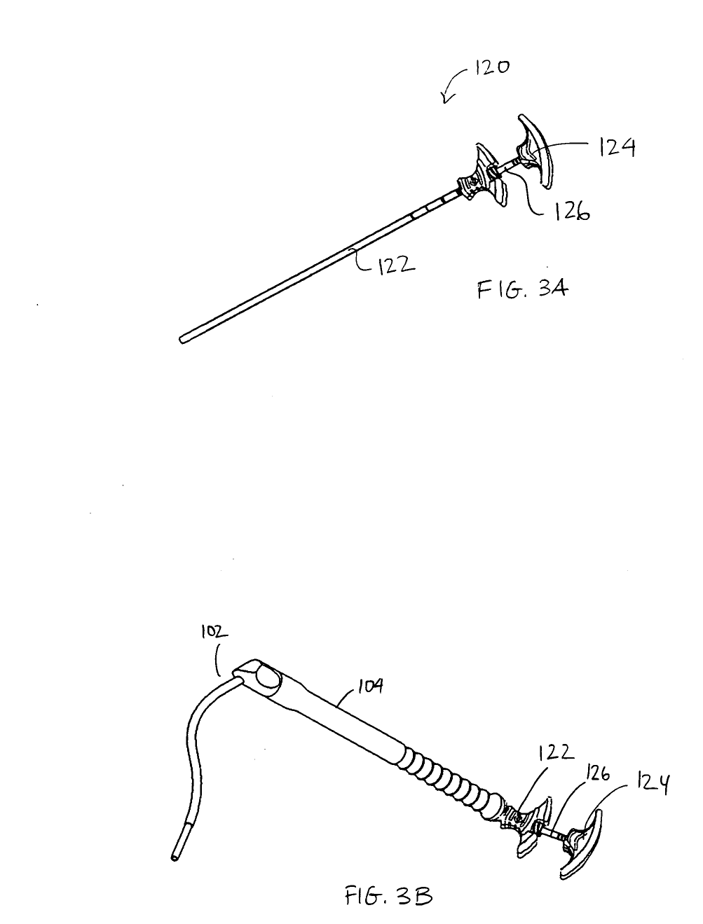

Referring to FIGS. 3A and 3B, an implementation of a plunging assembly 120 that can be used to advance the material through the barrel 104 and nozzle assembly 102 and into the cavity 116 is shown. The plunging assembly 120 includes a cartridge 122 and a plunger 124. The cartridge 122 has a hollow interior that can be loaded with the material. The plunger 124 includes a plunging rod 126 that has an outer diameter configured to fit within the hollow interior of the cartridge 122.

The loaded cartridge 122 is inserted into the barrel 104 as shown in FIG. 3B. In one implementation, the cartridge 122 is at least long enough so that when inserted into the barrel 104, the cartridge 122 extends the entire length of the interior of the barrel 104. The plunger 124 is inserted into the cartridge 122 and the plunging rod 126 is pushed through the interior of the cartridge 122 to advance the material contained therein. The material advances out of the cartridge 122 and either into the barrel 104 and then into the nozzle assembly 102, or directly into the nozzle assembly 102.

The nozzle assembly 102 can be primed, i.e., pre-loaded with the material, so that when the plunging rod is pushed through the cartridge 122 and the material advances from the cartridge 122 into the nozzle assembly 102, the pre-loaded material in the nozzle assembly 102 is forced out of the distal end 110 and into the cavity 116 (FIG. 2).

Referring again to FIGS. 1A-C and 2, including the flexible tube 108 in the nozzle assembly 102 can make it easier for an operator of the cavity-filling assembly 100 to maneuver the device during a cavity filling operation. For example, a C-arm imaging device for fluoroscopy may be used to provide instantaneous imaging data to the operator to assist the operator in filling a cavity 116 with the material. Maneuvering a cavity-filling device while constrained by the clearance between the C-arm imaging device and the patient's body can be difficult. The flexible tube 108 allows flexibility in positioning the cavity filling assembly 100 to accommodate the clearance constraint. Including a rigid distal end 110 at the end of the flexible tube 108 can make it easier for the operator to control the distal end 110 within the cavity 116, particularly while discharging the material into the cavity 116. The distal end 110 may include different configurations to further control dispensing of the cavity filling material.

Other implementations of the cavity-filling assembly 100 are possible, including a cavity-filling assembly 100 with a differently configured nozzle assembly 102. In one implementation, the nozzle assembly 102 can be a flexible tube 108 without a rigid distal end 110, i.e., an entirely flexible nozzle assembly 102. In another implementation, the flexible tube 108 can be replaced with a rigid tube. That is, the nozzle assembly 102 can be entirely rigid.

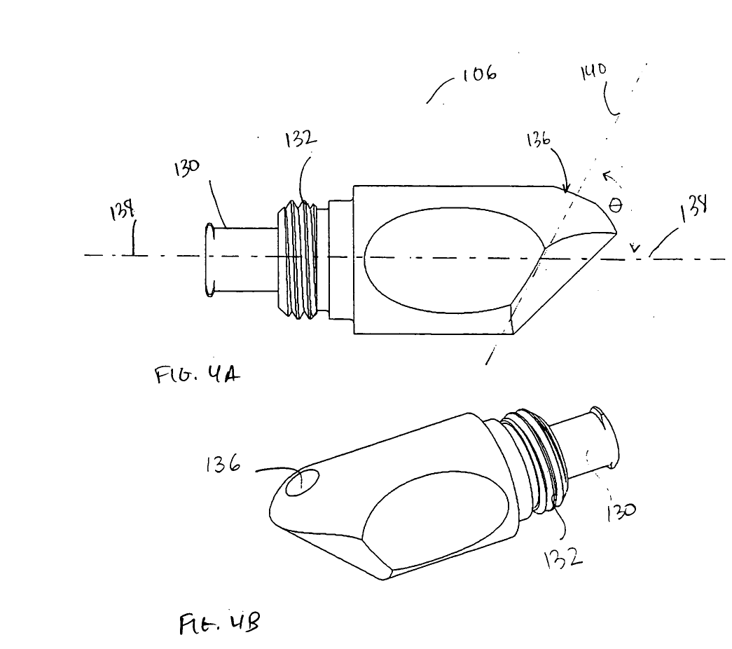

Referring to FIGS. 4A and 4B, the connector 106 included in the cavity-filling assembly 100 shown in FIGS. 1A-C is shown. In one implementation, the connector 106 includes threads 132 to mate with threads 134 formed in the interior of the barrel 104. For example, referring again to FIG. 1C, the fitting 112 of the barrel 104 is shown, which includes a threaded interior surface 134 that can mate with the threads 132 formed on the connector 106. A luer fitting 130, in addition to threads 132, can be present on the connector 106 for ease of pre-loading or priming the nozzle assembly 102 with cavity filling material. In another implementation, a luer fitting 130 can be used to connect to the barrel 104. The connector 106 includes a connector face 136 that receives or attaches to the flexible tube 108 of the nozzle assembly 102. In one implementation, the tubing is chemically or thermally bonded to the connector 106. In another implementation, the tubing can be threaded or snap-fit with the connector 106 to allow the operator to easily change the flexible tube 108 for different applications, for example, to use a specially configured distal end. In the implementation shown, the connector face 136 is angled, such that a central axis of the barrel 104 attached to the luer fitting 130 or the threads 132 is at an angle θ to a central axis of a flexible tube 108 attached to the connector face 136, as shown by the intersection of lines 138 and 140 at the angle θ. The connector face 136 can be angled such that the angle θ can range from approximately 0° to 90°, depending on the desired orientation of the flexible tube 108 relative to the barrel 104. In one implementation, an angle of 45° is used.

Other embodiments of the connector 106 can be used. For example, a different fitting can be used in place of the luer fitting 130, e.g., a snug-fit, snap-on fit, or tapered fitting. The threads 132 formed on the connector 106 are optional.

Referring again to FIGS. 1A-C, the barrel 104 provides an extension from the nozzle assembly 102 that an operator can use as a handle during a cavity filling operation. The barrel 104 also acts to distance the operator's hands from the patient's body, which can also act to keep the operator's hands outside of a potentially harmful imaging field. For example, as described above, in a kyphoplasty operation a C-arm imaging device is typically used during the cavity filling operation to provide instantaneous imaging information to the operator. The barrel 104 operates as an extender that keeps the operator's hands and arms out of the imaging field created by the imaging device. The length of the barrel 104 and the length of the flexible tubing 108 can be designed based on how far the operator's hands must be from the imaging field. Further, there may be a relatively small clearance between the C-arm imaging device and the patient's body. The connector 106 between the nozzle assembly 102 and the barrel 104 allows the barrel 104 to be angled relative to the nozzle assembly 102 to achieve preferred flow characteristics. The flexible tubing 108 allows for low clearance. In one implementation, the barrel and plunging assembly can be a syringe.

The cavity-filling assembly 100 shown in FIGS. 1A-1C is a two-part assembly including the nozzle assembly 102 and the barrel 104. In another implementation, the cavity-filling assembly 100 can be a unitary apparatus wherein the nozzle assembly 102 and the barrel 104 are permanently affixed to one another. In this implementation, the cavity-filling assembly 100 can be primed by pre-loading the unitary cavity-filling assembly 100 with a material for injection into a cavity 116 (FIG. 2). The material can be advanced through the cavity-filling assembly 100 and out of the distal end 110 by plunging the interior of the pre-loaded barrel 104 directly. That is, rather than inserting a cartridge 122 loaded with the material into the barrel 104, the barrel 104 is loaded with the material and plunged using a plunging assembly 120 directly.

In either the two-part or unitary cavity-filling assembly 100 implementations, the barrel 104 can be configured to have substantially the same inner diameter as the nozzle assembly 102, so as to maintain a constant pressure across the cavity-filling assembly 100 as the material is advanced therethrough. Alternatively, the barrel 104 can be configured to have a larger inner diameter than the nozzle assembly 102, to increase the amount of material that can be preloaded into the cavity-filling assembly 100, thereby minimizing the number of loading iterations necessary to fill a cavity 116 (FIG. 2).

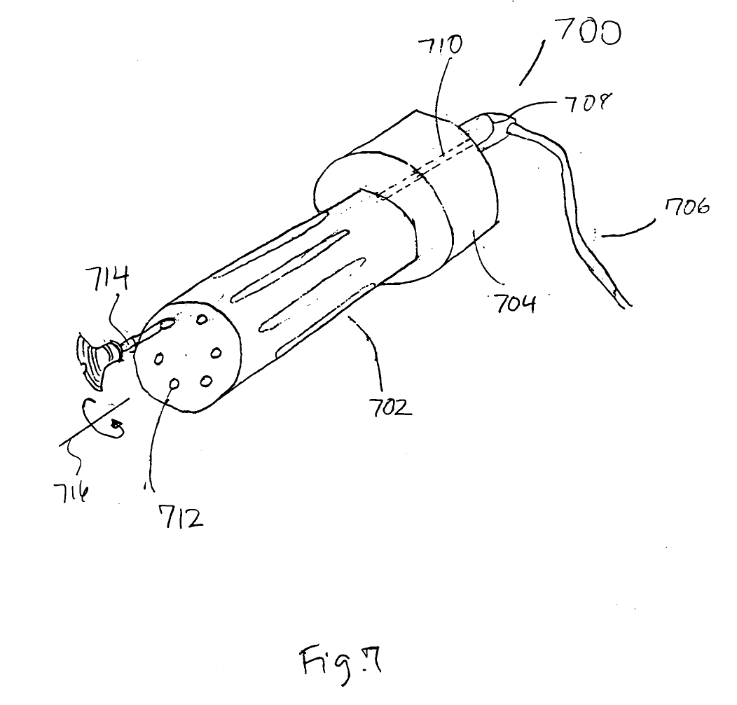

A cavity filling operation may require that the nozzle assembly 102 and barrel 104 be reloaded (either directly or via a cartridge 122) with material more than once in order to completely fill the cavity. As shown in FIG. 7, in one implementation, a barrel 700 can include multiple chambers 712, each of which can be preloaded with the material. The chambers 712 can be arranged similar to the chambers in a revolver, in that the barrel 700 can be rotated about an axis 716 within a sleeve 704 to engage a chamber 712 with a connector 708 of the nozzle assembly 706 by way of an exit port 710. In the implementation depicted, each chamber 712 is configured to receive a pre-loaded cartridge 714. The material in the chambers 712 can be discharged one by one into the nozzle assembly 706, e.g., by rotating the barrel 702 and plunging the cartridges 714. In another implementation, each of the multiple chambers 712 can be loaded with material directly (i.e., without using cartridges) and the material discharged from the chambers 712 using a plunging assembly as described above. The implementation shown includes 6 chambers 712, however, any number of chambers can be included.

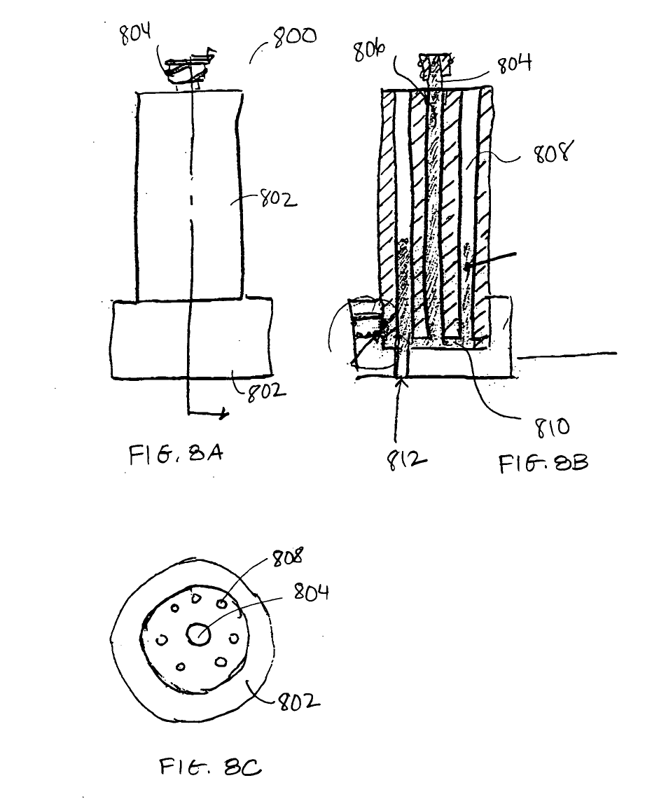

FIGS. 8A-C show an alternative implementation of a multi-chambered barrel assembly 800. The barrel assembly 800 includes a barrel 802 rotatable about a longitudinal axis within a sleeve 802. FIG. 8B shows a longitudinal cross-sectional view of the barrel assembly 800. The barrel 802 includes a central bore 806 having an inlet port 804. Multiple chambers 808 are arranged about the central bore 806, as shown in the cross-sectional view in FIG. 8C. The chambers 808 can be filled with material via the central bore 806. That is, material is injected into the central bore 806 by the inlet port 804. A reservoir 810 in the sleeve 802 redirects the material into the chambers 808. The barrel 802 can be rotated within the sleeve 802 to engage a chamber 808 with a connector of a nozzle assembly by way of an exit port 812. The material in the chambers 808 can be discharged one by one into the nozzle assembly, e.g., by rotating the barrel 802 and plunging the chambers 808. The implementation shown includes 7 chambers 808, however, any numbers of chambers can be included.

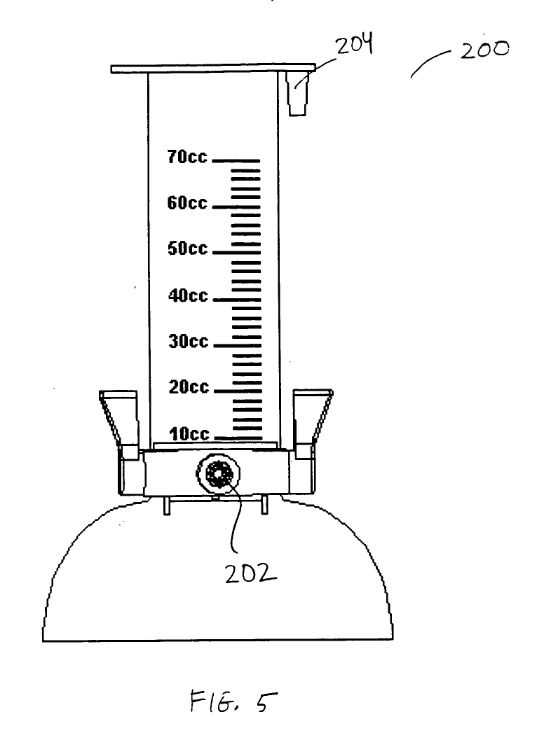

In one implementation where the barrel 104 is loaded with the material directly rather than using a cartridge 122, the distal end of the barrel 104 used to load the material into the barrel 104 is configured to mate with a mixer used to mix and contain the material. Referring to FIG. 5, an example of a mixer 200 is shown. In one implementation, the mixer 200 can be a Kyphon® Mixer available from Kyphon Inc. The cavity filling material can be mixed directly in the mixer 200, for example, a bone filling cement can be mixed by the addition of a liquid to a cement powder. The mixer 200 can include a dispenser 202 near the base that is configured to mate with the distal end of the barrel 104. For example, the dispenser 202 can include a male portion of a luer fitting, and the distal end of the barrel 104 can include a female portion of a luer fitting that is configured to mate with the dispenser 202. A plunger 204 included within the mixer 200 can be depressed to urge the material from the mixer 200 and through the dispenser 202 to load the cartridge 122. If the barrel 104 includes multiple chambers, then each chamber may include a female portion of a luer fitting to mate with the dispenser 202, and each chamber loaded separately; or a main loading chamber can be connected to a manifold to simultaneously load all the chambers.

In one implementation, the cartridge 122 can be a KyphX® Bone Filler Device available from Kyphon Inc. The cartridge 122 (or nozzle) can include a fitting configured to mate with a fitting on a dispenser of a mixer, such as the mixer 200 shown in FIG. 5. For example, the cartridge 122 can include a female portion of a luer fitting that is configured to mate with a male portion of a luer fitting formed on the dispenser 202 of the mixer 200. The cartridge 122 is attached to the dispenser 202 to load the cartridge 122 with the material.

Referring again to FIGS. 3A-B, in one implementation, the plunging assembly 120 is included with the KyphX® Bone Filler Device available from Kyphon Inc. The plunging assembly 120 is configured to plunge the cartridge 120 (or nozzle) of the KyphX® Bone Filler Device available from Kyphon Inc. Alternatively, if the barrel 104 of the cavity-filling assembly 100 is to be loaded directly with the material (rather than via a loaded cartridge), then the plunging assembly 120 can be adapted to plunge the barrel 104, for example, to accommodate the interior diameter and length of the barrel 104.

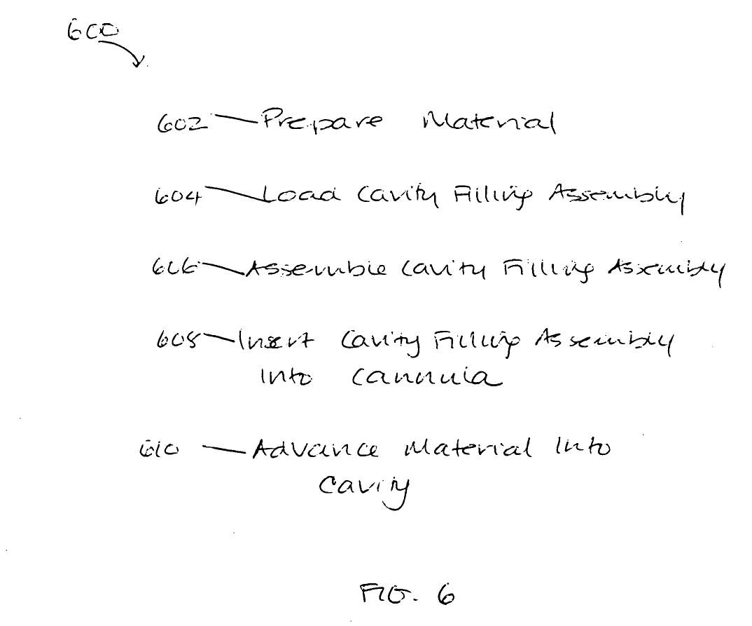

FIG. 6 shows a process 600 for using the cavity filling assembly 100 to fill a cavity in a patient's body. Referring now to FIGS. 1A-C, 2, 3A-B and 6, the material that will be used to fill the cavity is prepared (step 602). The cavity filling material can differ depending on the cavity to be filled, for example, in a kyphoplasty procedure a bone filling cement, such as polymethylmethacrylate (PMMA) or a calcium phosphate bone substitute, can be used. Preparing the material may include mixing a powder with a liquid and stirring until the cement mixture reaches a desired consistency. The cavity filling assembly 100 is loaded with the cavity filling material (step 604). For example, in an implementation including a separate nozzle assembly 102 and barrel 104, the nozzle assembly 102 can be primed by loading the nozzle assembly 102 with the cavity filling assembly. The barrel 104 can also be loaded with the material, in a configuration that loads the barrel 104 directly rather than via a cartridge 122.

The cavity filling assembly 100 is assembled (step 606), if necessary. For example, if the cavity filling assembly includes a separate nozzle assembly 102 and barrel 104, then the barrel 104 and nozzle assembly 102 can be joined by the connector 106. The cavity filling assembly 100 is inserted into a cannula 114 that has been positioned within the cavity 116 in the patient's body (step 608). In one implementation, one or more surgical balloons are first inflated within the cavity 116 to restore the cavity 116 to an original shape or to create or enlarge the cavity 116. The surgical balloons are removed, and optionally material, such as bone, may be removed from the cavity 116. Once the cavity is prepared, the cavity filling assembly 100 is inserted into the cannula 114.

The material is advanced from the cavity filling assembly 100 into the cavity 116 (step 610). This step can include, if necessary, inserting one or more cartridges 122 into the barrel 104 to load the barrel 104 with the material, i.e., in a configuration where the barrel 104 is not directly loaded with the material. A plunging assembly 120 is positioned to either plunge the barrel 104 or a cartridge 122 contained within the barrel 104. The plunging rod 122 is advanced into the barrel 104 or cartridge 122 to advance the material through the cavity filling assembly 100 and out the distal end 110 that is positioned within the cavity 116.

The operator can control the advancement of the plunging rod 122, thereby controlling the advancement of the material into the cavity 116. In one implementation, the material is advanced into the cavity 116 at a low pressure. If the inner diameters of the components of the cavity filling assembly 100, i.e., of the nozzle assembly 102 and barrel 104 or cartridge 122, are substantially the same, then the advancement of the plunging rod 122 is directly proportional to the advancement of the material from the distal end 110 of the nozzle assembly 102 into the cavity 116 (i e., an approximate one-to-one ratio). In one implementation, the diameters of the components of the cavity filling assembly 100 are uniform and allow for no drop-off or build-up of pressure in the cavity filing material. Further, the operator can retract the nozzle assembly 102 from the cannula 114 while advancing the plunging rod 122, thereby pulling the distal end 110 out of the cavity 116 as the cavity 116 is filled with the material. That is, the front portion of the cavity can be filled first with the viscous material to ensure a solid, compact fill with no air gaps. The one-to-one ratio of the advancement of the plunging road to the advancement of the material, and the ability to retract the nozzle assembly 102 from the cavity 116 during the filling operation provides improved control to the operator over the cavity filling procedure.

The cavity filling assembly 100 can be formed from any suitable material or combination of materials. The materials selected must be compatible with the cavity filling material that will be advanced through the assembly 100. The materials used for the flexible tubing 108 should also provide kink resistance, burst resistance and thermal resistance. The flexible tube 108 can be formed from polyamides, nylon, Teflon®, or nylon with a Teflon lining, Pebax®, PEEK, polypropylene, polyethylene, PTFE, FEP, PFA, Radel® R polyphenylsulphone and the distal end can be formed from stainless steel. In a preferred embodiment, the flexible tubing can be formed of Pebax® with an inner stainless steel coil reinforcement. Examples of material for the barrel 104 and connector 106 include nylon, polyethylene, polycarbonate, aluminum, Radel® R polyphenylsulphone or stainless steel.

A number of embodiments of the invention have been described. Nevertheless, it will be understood that various modifications may be made without departing from the spirit and scope of the invention. The logic flow depicted in FIG. 6 does not require the particular order shown, or sequential order, to achieve desirous results, and the steps of the invention can be performed in a different order. Accordingly, other embodiments are within the scope of the following claims.

1. An apparatus comprising:
a flexible tube having a rigid first end, a second end, and a lumen extending from the first end to the second end and operable to hold a material for conveyance through the rigid first end into a cavity;
a barrel in fluid communication with the second end of the flexible tube, the barrel including a lumen configured to receive material for delivery into the flexible tube; and
a plunger configured to advance material through the lumen in the barrel and into the flexible tube, where there is substantially a one to one ratio of advancement of the plunger to a volume of material advanced from the rigid first end of the flexible tube.

2. The apparatus of claim 1, further comprising:
a cannula having a first end configured to receive the first end of the flexible tube and having a second end configured to provide the first end of the flexible tube access to the cavity.

3. The apparatus of claim 1, wherein:
the lumen of the flexible tube is pre-loaded with the material; and
the plunger is operable to advance material through the lumen in the barrel into the flexible tube and advance the pre-loaded material in the flexible tube through the lumen in the flexible tube and into the cavity.

4. The apparatus of claim 1, further comprising:
a connector connecting the flexible tube to the barrel, where the flexible tube is connected to the barrel such that a longitudinal axis of the barrel is at an angle in the range of approximately 0° to approximately 90° to a longitudinal axis of the flexible tube.

5. The apparatus of claim 4, wherein the angle is substantially 40° to 50°.

6. The apparatus of claim 4, wherein the connector is a luer fitting.

7. The apparatus of claim 6, wherein:
the barrel includes threads on an end of the lumen; and
the connector further comprises threads configured to mate with the threads on the barrel.

8. The apparatus of claim 4, wherein:
an assembly of the flexible tube connected to the barrel is pre-loaded with the material; and
the plunger is operable to advance at least some of the pre-loaded material into the cavity.

9. The apparatus of claim 1, the barrel further comprising:
a plurality of lumens disposed in the barrel and configured to receive material for delivery into the flexible tube,
wherein, the barrel is rotatable about a longitudinal axis of the barrel and where each lumen of the plurality of lumens is preloaded with material, such that once a lumen has been emptied into the flexible tube by action of the plunger, the plunger can be withdrawn, the barrel can be rotated and the plunger can engage a next lumen to fill the flexible tube.

10. The apparatus of claim 1, further comprising:
a cartridge configured to receive material for delivery into the flexible tube,
wherein, the lumen is configured to receive the cartridge and to receive the material via the cartridge, and the plunger is configured to advance material through the lumen in the barrel via advancement through the cartridge.

11. An apparatus comprising:
a flexible tube having a rigid first end, a second end, and a lumen extending from the first end to the second end and operable to hold a material for conveyance through the rigid first end and into a cavity;
a barrel in fluid communication with the second end of the flexible tube, the barrel including a lumen configured to receive material for delivery into the flexible tube, where the barrel's lumen has a diameter substantially the same as a diameter of the flexible tube's lumen; and
a plunger configured to advance material through the lumen in the barrel and into the flexible tube.

12. The apparatus of claim 11, further comprising:
a cannula having a first end configured to receive the first end of the flexible tube and having a second end configured to provide the first end of the flexible tube access to the cavity.

13. The apparatus of claim 11, wherein:
the lumen of the flexible tube is pre-loaded with the material; and
the plunger is operable to advance material through the lumen in the barrel into the flexible tube and advance the pre-loaded material in the flexible tube through the lumen in the flexible tube and into the cavity.

14. The apparatus of claim 11, further comprising:
a connector connecting the flexible tube to the barrel, where the flexible tube is connected to the barrel such that a longitudinal axis of the barrel is at an angle in the range of approximately 0° to approximately 90° to a longitudinal axis of the flexible tube.

15. The apparatus of claim 14, wherein the angle is substantially 40° to 50°.

16. The apparatus of claim 14, wherein the connector is a luer fitting.

17. The apparatus of claim 16, wherein:
the barrel includes threads on an end of the lumen; and
the connector further comprises threads configured to mate with the threads on the barrel.

18. The apparatus of claim 14, wherein:
an assembly of the flexible tube connected to the barrel is pre-loaded with the material; and
the plunger is operable to advance at least some of the pre-loaded material into the cavity.

19. An apparatus comprising:
a flexible tube having a first end and a second end, and a lumen extending from the first end to the second end and operable to hold a material for conveyance through the first end;
a barrel in fluid communication with the second end of the flexible tube, the barrel including a lumen configured to receive material for delivery into the flexible tube; and
a plunger configured to advance material through the lumen in the barrel and into the flexible tube.

20. The apparatus of claim 19, wherein a diameter of the lumen of the barrel is greater than a diameter of the lumen of the flexible tube.

21. The apparatus of claim 19, further comprising:
a cannula having a first end configured to receive the first end of the flexible tube and having a second end configured to provide the first end of the flexible tube access to the cavity.

22. The apparatus of claim 19, wherein:
the lumen of the flexible tube is pre-loaded with the material; and
the plunger is operable to advance material through the lumen in the barrel into the flexible tube and advance the pre-loaded material in the flexible tube through the lumen in the flexible tube and into the cavity.

23. An apparatus comprising:
a flexible tube having a rigid first end, a second end, and a lumen extending from the first end to the second end and operable to hold a material for conveyance through the rigid first end into a cavity;
a barrel in fluid communication with the second end of the flexible tube, the barrel including a lumen configured to receive a cartridge pre-loaded with material for delivery into the flexible tube;
a cartridge configured for insertion into the barrel and including a lumen configured to receive material for delivery into the flexible tube; and
a plunger configured to advance material through the lumen in the cartridge and into the flexible tube, such that there is substantially a one to one ratio of advancement of the plunger to a volume of material advanced from the rigid first end of the flexible tube.

24. The apparatus of claim 23, further comprising:
a cannula having a first end configured to receive the first end of the flexible tube and having a second end configured to provide the first end of the flexible tube access to the cavity.

25. The apparatus of claim 23, further comprising:
a connector connecting the flexible tube to the barrel, where the flexible tube is connected to the barrel such that a longitudinal axis of the barrel is at an angle in the range of approximately 0° to approximately 90° to a longitudinal axis of the flexible tube.

26. The apparatus of claim 25, wherein the angle is substantially 40° to 50°.

27. The apparatus of claim 25, wherein the connector is a luer fitting.

28. The apparatus of claim 27, wherein:
the barrel includes threads on an end of the lumen; and
the connector further comprises threads configured to mate with the threads on the barrel.

29. The apparatus of claim 23, further comprising:
a plurality of cartridges configured for insertion into the barrel, each cartridge including a lumen configured to receive material for delivery into the flexible tube; and
wherein the barrel further comprises a plurality of lumens configured to receive a plurality of cartridges, the barrel is rotatable about a longitudinal axis of the barrel and where each lumen can be loaded with a cartridge pre-loaded with material, such that once a cartridge has been emptied into the flexible tube, the barrel can be rotated and a next cartridge used to fill the flexible tube.
